(19) United States
(12) Patent Application Publication
(10) Pub. No.: US 2004/0202694 A1
(43) Pub. Date: Oct. 14, 2004

(54) Embolic occlusion of uterine arteries

(75) Inventors: Fred H. Burbank, Laguna Niguel, CA (US); Greig Altieri, Laguna Beach, CA (US); Michael L. Jones, San Clemente, CA (US)

Correspondence Address: Edward J. Lynch, DUANE MORRIS, One Market, Spear Tower, Suite 2000, San Francisco, CA, 94105, US

(73) Assignee: Vascular Control Systems, Inc.

(21) Appl. No.: 10/411,690

(22) Filed: Apr. 11, 2003

(51) Int. Cl. A61F 6/06

(52) U.S. Cl. 424/430

(57) ABSTRACT

A treatment procedure is disclosed which involves the short term, non-permanent occlusion of the patient's blood vessels by depositing a bioabsorbable embolic mass within the patient's blood vessel. The procedure is particularly suitable for treating uterine disorders by occluding a patient's uterine arteries. A therapeutically effective time period for occlusion of a uterine artery is from about 0.5 to about 48 hours, preferably about 1 to about 24 hours, with occlusion times of about 1 to about 8 hours being suitable in many instances. The embolic mass may bioabsorbable particulate with minimum transverse dimensions of about 100 to about 2000 micrometers, preferably about 300 to about 1000 micrometers. The particulate may be a polymeric material formed of polylactic acid, polyglycolic acid or copolymers thereof, or a swellable copolymer of lactic acid and polyethylene glycol. The embolic material may be delivered to an intracorporeal site as a biocompatible solution containing a solute which is relatively insoluble in a water based fluid and a solvent which is relatively soluble in the water based fluid, where the solute forms the embolic mass which occludes or partially occludes a body lumen or fills or partially fills a body cavity.

### FIELD OF THE INVENTION

The present invention relates generally to the treatment of uterine disorders which involve occluding one or more of the patient's uterine arteries, and more particularly to the non-permanent, embolic occlusion of the patient's uterine artery or arteries.

### BACKGROUND OF THE INVENTION

Hysterectomy (surgical removal of the uterus) is performed on about 600,000 women annually in the United States. For approximately 340,000 women, hysterectomy is probably the best current therapeutic choice for the treatment of their diseases (uterine cancer, endometriosis, menorrhagia, and prolapse). For approximately 60,000 women with dysfunctional uterine bleeding (abnormal menstrual bleeding that has no discrete anatomic explanation such as a tumor or growth), newer endometrial ablation techniques may be an alternative to hysterectomy. For approximately 200,000 women with benign but symptomatic (excessive bleeding, pain, and “bulk” sensations) muscular tumors of the uterus, known as leiomyoma or fibroids, newer treatment methods have been developed which may spare these women a hysterectomy, as well.

Hysterectomy for treating uterine fibroid disorders, though effective, has many undesirable characteristics. The undesirable characteristics of hysterectomy are well know and can include a mortality rate of about 0.5 deaths per 1000 hysterectomies, injury to adjacent organs (the bladder, the ureters, and bowel), a hospital stay of approximately one week, five to six weeks of slow recovery to normal activity, substantial medical expenses, increased risk of cardiovascular disease, reduced libido, and depression and anxiety.

Commonly, a diagnosis of uterine fibroids involves the presence of multiple fibroids, often averaging ten fibroids or more per afflicted uterus. Moreover, it is frequently difficult to know which fibroid is causing symptoms to the patient (bleeding, pain, and bulk effects on adjacent organs). Furthermore, fibroids occur at different layers in the uterus, submucosal fibroids can occur adjacent to the lining of the uterus, intramural fibroids can occur in the myometrium, and subserosal fibroids may occur adjacent to the outer layer of the uterus. Generally, only subserosal fibroids will be directly observed from the peritoneal cavity, and only submucosal fibroids will be observed from the endometrial surface of the uterus. Fibroids deep within the wall of the uterus are poorly visualized from either surface. Finally, since fibroids come in all sizes, only the larger fibroids will be seen in any case.

Clearly, the strategy of identifying which individual fibroid or group of fibroids is causing symptoms is difficult, finding that fibroid, and then either removing or destroying that individual fibroid is a rather complex strategy. It is therefore easy to understand why the hysterectomy is such a common surgical choice. With hysterectomy, all uterine fibroids are removed in one stroke.

Ravina et al. in 1995 demonstrated that uterine fibroids could be treated using a non-surgical, intravascular therapy, specifically comprising bilateral intraluminal occlusion of the uterine arteries (Ravina et al., “Arterial Embolization to Treat Uterine Myomata”, Lancet Sep. 9, 1995; Vol. 346; pp. 671-672, incorporated by reference in its entirety herein). This technique is now commonly known as “uterine artery embolization”. Uterine artery embolization can also be effectively used to control uterine bleeding from a variety of sources.

The technique uses standard interventional radiology angiographic techniques and equipment. The femoral artery is accessed by a conventional Seldinger technique and a delivery catheter is advanced through the femoral artery into the right and left uterine arteries where embolic material is deposited to occlude the uterine arteries. Initially, polyvinyl alcohol particles (PVA) were used as the embolic media, but other embolic media can be used including metallic coils such as those used for aneurysm treatments (See U.S. Pat. Nos. 4,994,069, 5,226,911, and 5,549,824, all of which are incorporated in their entireties herein), or particles such as GELFOAM pledgets, available from Upjohn, Kalamazoo, Mich., or IVALON particles, available from Boston Scientific). Following the occlusion, the delivery catheter and other devices are removed from the patient and the arterial puncture site is held with manual pressure for about fifteen minutes. While post-procedural pain is often significant, the patient is typically fully recovered in a number of days.

One of the key features for treating fibroids with uterine artery embolization is the fact that fibroids live a tenuous vascular life with very little ability to recruit a new blood supply from the host when the primary blood supply is compromised. The uterus on the other hand has a dual (or redundant) blood supply; the primary blood supply is from the bilateral uterine arteries, the secondary blood supply from the bilateral ovarian arteries. Consequently, when both uterine arteries are occluded, i.e., bilateral vessel occlusion, the uterus and the fibroids contained within the uterus are both deprived of their blood supply. However, as demonstrated by Ravina et al., the effect on the fibroids is greater than the effect on the uterus. In most instances, the fibroids wither and cease to cause clinical symptoms.

While uterine artery embolization has been successful, its use has not been widespread because the physicians who do the procedure are interventional radiologists, who do not usually take care of gynecology problems. The physicians who take care of gynecology problems usually do not possess the training and equipment necessary to perform catheter-based embolization. Accordingly, much fewer uterine artery embolizations are performed than hysterectomies for symptomatic uterine fibroids.

Most of the current treatments offered to women for fibroid treatment or uterine bleeding focus on permanent or near permanent occlusion methods for the uterine artery. For example, embolizing with PVA particles causes uterine artery occlusion for 6 months or more; embolizing with stainless steel coils causes permanent occlusion; embolizing with Gelfoam occludes for 3 to 4 weeks before degradation of the embolic particles and additionally causes sever inflammation; surgical ligation with metal vascular clips occlude permanently; and surgical ligation with RF ablation results in permanent occlusion.

The prior art devices and methods are therefore aimed at long term or permanent occlusion of the uterine artery, which is not suitable for women of child bearing age who may desire to bear additional children. These patients of childbearing age are frequently the patients who suffer most dramatically from uterine myomata. While there have been reports of women who have undergone uterine artery embolization with PVA particles and who have subsequently become pregnant and deliver normal babies, the fetus must be nourished by a uterus deprived of blood flow through the uterine arteries. Women who have undergone uterine artery embolization have also experienced premature menopause due to ovarian failure.

Recently, Burbank et al. in co-pending applications Serial No. 09/908,815, filed Jul. 20, 2001 and Serial No. 10/107,810, filed Mar. 28, 2002 (both assigned to the present assignee and which are incorporated herein by reference) disclosed temporarily occluding a female patient's uterine arteries for fibroid and uterine bleeding by clamping or otherwise mechanically applying pressure to the uterine artery.

There still remains a need in the art for improvements in methods, processes, and techniques for uterine artery embolization without the undesirable side effects of the prior embolic procedures.

### SUMMARY OF THE INVENTION

The invention is directed to treating a human or other mammalian patient by occluding one or more of the patient's arteries with a bioabsorbable, short lived embolic material for a therapeutically effective time period. The occluding mass of embolic material may be deployed as a bolus or may be formed in situ. The bioabsorbable, short lived, embolic material is delivered into the patient's artery to occlude the artery and, preferably, blood flow through the artery is monitored during the therapeutically effective time period to ensure that such blood is reestablished at the end of the time period. The treatment is particularly suitable for uterine disorders such as fibroids, dysfunctional uterine bleeding (DUB), post partum hemorragh (PPH) and bleeding from caesarian section surgery.

In order to ensure that the thrombus is formed well enough to occlude the uterine artery in accordance with the processes of the present invention, hemostasis should be maintained for at least about 0.5 hours but not more than about 48 hours, preferably about 1 hour to about 24 hours and most preferably about 1 to about 8 hours. The mass of embolic material which initiates formation of the thrombus, described in greater detail below, must stay in place for sufficient time to provide for the death of the fibroid cell line, stop the uterine bleeding or otherwise effectively treat the patient's uterine disorder. After this initial period to initiate and maintain the formation of a thrombus in the artery, the embolic mass delivery system can be removed or dispersed from the occluded arterial site.

The occluding embolic mass may be formed of particulate material having a minimum transverse dimension of about 100 to about 2000 micrometers, preferably about 300 to about 1000 micrometers. Particles may generally be spherically shaped, but particles with diameters larger than about 400 micrometers should have an aspect ratio (length to diameter) of at least 1.5, preferably greater than 2 to facilitate delivery and to ensure proper orientation within the patient's uterine artery.

The embolic material may be formed of non-swellable, bioabsorbable polymeric materials such as polylactic acid, polyglycolic acid, polycaprolactone and copolymers, blends and mixtures thereof, or swellable (hydratable) bioabsorbable polymeric materials such as a copolymer of lactic acid and polyethylene glycol. A particularly suitable swellable, bioabsorbable particulate which is formulated and sold by Birmingham Inc. is a copolymer of 70% (by wt.) polylactic acid (PLA) and 30% (by wt.) polyethylene glycol (PEG) 8000. Other suitable weight ratios of PLA to PEG may be utilized from about 95:5 to about 50:50. The molecular weight of the PEG can effect dissolution time with higher molecular weights (e.g. 10,000 to 20,000) taking longer to dissolve. The 70% PLA/30%PEG 8000 copolymer swells quickly and dissolves within 14 hours. The larger particles or pellets, e.g. minimum transverse dimension above about 400 micrometers, are preferably formed of swellable polymeric material which can be deposited in a more proximal location. Because the pellets are swellable in an aqueous-based fluid such as blood, they expand in situ to provide a more secure fit within the arterial lumen and also ensure complete blockage of the artery.

The embolic mass may also be in the form of a very viscous liquid or a gel-like mass such as polyethylene glycol and methyl cellulose.

The embolic material which occludes the patient's uterine arteries may also be delivered as a solution of the occluding material which forms an occluding mass when deployed within the patient's uterine artery. The biocompatible and bioabsorbable material should be water insoluble and preferably is a polymeric material such as polylactic acid, polyglycolic acid, polycaprolactone or copolymers, blends and mixtures thereof, is dissolved at least in part in a water soluble biocompatible solvent such as dimethyl sulfoxide (DMSO) or other suitable biocompatible solvent. The polymer material is not soluble in the water based body fluid but the solvent is, so when a bolus of the solution is injected into the patient's artery, the solvent is quickly taken up by the blood stream or other body fluid and the insoluble polymeric material remains, forming in-situ an occluding mass. Usually the in-situ formed polymeric mass is porous but nonetheless occludes the patient's uterine artery in which it is disposed. The amount of solute ranges from about 1 to about 35% (by wt.), preferably about 2 to about 20% (by wt.) depending upon the composition of the solute and solvent.

While described herein with reference to occluding a patient's uterine artery, the solution of water soluble, preferably bipolar solvent and water insoluble polymeric solute which is dissolved in the solvent may be used at other intracorporeal sites in situations in which a short-lived, bioabsorbable polymeric mass is needed at an intracorporeal site for a variety of reasons such as mechanical support, drug delivery, cavity fill and the like. Other non-dissolved components may be incorporated into the solution for other purposes. For example, a particulate with a drug or other therapeutic or diagnostic agent incorporated therein which is insoluble in the solution or water may be delivered with the solution so that the bioabsorbable structure formed by the polymeric solute supports the particulate having incorporated one or more therapeutic or diagnostic agents for delivery over a period of time which is governed by the bioabsorption of the polymeric solute.

The absorption rate of the occluding embolic mass and the enzymatic breakdown rate of thrombus are such that the uterine arteries are effectively occluded for the therapeutic period of time, which should be less than 48 hours and typically less than 24 hours. The occlusion by the embolic mass need not be complete. The embolic mass may slow blood flow through the artery sufficiently, for a blood clot to form in the uterine artery system feeding the patient's uterus and any fibroids associated with the uterus. Once the blood clot is formed, the clot itself can further slow or stop blood flow through the uterine artery. At the end of the therapeutic period of time, the occluding embolic mass has been sufficiently absorbed and the thrombus sufficiently lysed to permit reestablishment of blood flow through the uterine artery to the uterus. The thrombus lysing process can optionally be assisted by a systemic or localized administration of a thrombolytic agent, such as tPA, or the like, to accelerate the lysis, if the practitioner elects to do so.

The short term embolic occlusion of the uterine artery is sufficient to necrotize uterine myomata, to terminate uterine bleeding or perform other treatments for the patient's uterine disorder, without unnecessarily exposing adjacent tissues and anatomical structures to hypoxia or significant permanent damage. The intravascular methods for non-permanent uterine artery occlusion in accordance with the present invention allow for substantial improvements in safety and efficacy of this procedure over prior intravascular techniques and the procedure does not result in permanent damage to the patient's uterus.

Other objects, features, and attendant advantages of the present invention will become apparent from the following detailed description of the invention when taken in conjunction with the accompanying schematic drawing.

### BRIEF DESCRIPTION OF THE DRAWING

The FIGURE schematically illustrates a female patient's reproductive system including portions of a uterus, ovaries, fallopian tubes, vagina, and uterine and ovarian arteries after delivering a bolus of embolic material to both uterine arteries.

### DETAILED DESCRIPTION OF THE INVENTION

The treatments embodying features of the present invention for short term, non-permanent uterine artery occlusion are directed to the following events. Blood flow in the uterine artery is slowed or stopped by occluding the artery by delivery of one or more short lived embolic masses of bioabsorbable material to desired locations within the patient's uterine artery system. This stoppage of blood flow creates a clotting cascade within the artery in a fashion well known to those skilled in the art. Once blood flow has ceased, or has otherwise slowed sufficiently, within the uterine artery clotting or thrombus formation begins and very soon the blood vessel is filled with blood clots or thrombus. The uterine fibroids, and more particularly the cells of the uterine fibroids, suffer a near immediate death because of the cessation of blood flow to them. The uterus on the other hand becomes anoxic, but because the uterus is partially supplied by the ovarian arteries and other collateral arteries, the collateral circulation is adequate to keep the uterine tissues alive and allow for it to recover as the total blood flow to the uterus returns to normal.

The thrombus formed within the embolized occluded blood vessel is subjected to enzymatic activity which lyses the thrombus. This cycle is predictable and effective, and it can be assisted by the delivery of various thrombolytic agents such as tissue plasminogen activator (tPA) to the thrombus site. The thrombolytic agent delivery may be systemic or site specific with an intravascular catheter.

As will be readily appreciated by one of ordinary skill in the art, the present invention is not limited to the specific examples herein of mechanisms which are useful for occluding a uterine artery, and other suitable methods and devices are also within the spirit and scope of the present invention.

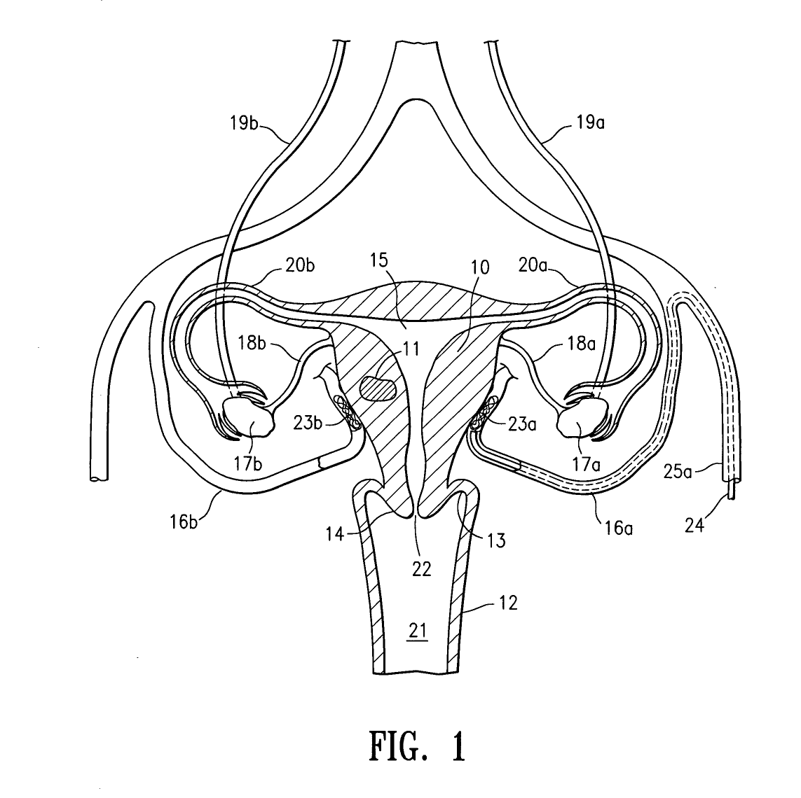

The FIG. 1 illustrates a portion of a female patient's reproductive system and adjacent anatomical structures. The drawing diagrammatically illustrates a uterus 10 which is afflicted with a fibroid tumor 11. The patient's vagina 12 includes the vaginal fornix 13 which surrounds the uterine cervix 14. The uterine cervix 14 leads to the uterine cavity 15. As discussed further herein, the left and right uterine arteries 16a, 16b respectively, extend to the uterus 10 and supply the uterus 10 and the fibroid 11 with oxygenated blood. The ovaries 17a and 17b are supported by ovarian ligaments 18a and 18b respectively extending out from the uterus 10 and are supplied oxygenated blood from ovarian arteries 19a and 19b respectively. The uterine arteries frequently extend through the ovarian ligaments 18a and 18b (not shown) to the ovaries 17a and 17b. The fallopian tubes 20a and 20b extend from the ovaries 17a and 17b respectively to the uterus 10 to direct ova, which are discharged from the patient's ovaries 17a and 17b. The patient's vagina defines the vaginal canal 21 extending to the uterine cervix 14 which defines the uterine os 22. Embolic masses 23a and 23b embodying features of the inventions are shown deposited within uterine arteries 16a and 16b respectively.

A delivery catheter 24 is percutaneously introduced into the patient's femoral artery 25a by conventional Seldinger techniques (not shown). Seldinger techniques usually include introducing a guiding catheter into the femoral artery through an introducer sheath and then advancing a delivery catheter through the inner lumen of the guiding catheter to the desired intravascular location. Preferably, the guiding catheter has a shaped distal tip to facilitate advancing the catheter through tortuous anatomy. The guiding catheter may itself be guided to the desired location by a conventional intravascular guide wire (not shown) having a shaped distal tip.

The internal diameters of uterine arteries will normally vary and typically are about 2 mm to about 5 mm prior to (upstream of) the first order branches of the artery at the uterus. The first order branches, typically, have internal diameters of less than 2 mm, with higher order branches having again smaller internal diameters.

Therefore, while the processes in accordance with the present invention can be performed on a uterine artery prior to the first order branches, the present invention also can be performed on higher order branches with smaller diameter blood vessels. Thus, reference to the uterine arteries herein includes the first order and higher order branches of the uterine artery system and the non-permanent occlusion of them.

The location of the occlusion generally will depend upon the size and shape of the occluding particles or mass. The larger particles or masses such as those greater than 1000 micrometers in maximum dimension will tend to occlude the uterine artery at a location leading to the patient's uterus, i.e. prior to the first order branches. Particles in the 500-1000 micrometer range will tend to occlude the portion of the uterine artery, the first order branches, leading to the helicine branches. Particles less than 500 micrometers will generally occlude the helicine branches and smaller diameter portion of the uterine artery.

As illustrated in the figure, the uterine arteries 16a, 16b extend generally laterally from the outer portions of the uterus in positions close to the vaginal fornix 14 which facilitates monitoring blood flow through the uterine arteries by a variety of methods and devices. However, one convenient method is described in previously mentioned co-pending applications Serial No. 09/908,815 and Serial No. 10/107,810, (which have been incorporated herein by reference) in which an intravaginal clamping device with one or more ultrasonic sensors are provided on the clamping members to detect blood flow. The clamping members are pressed against the wall of the patient's vaginal fornix 13 so that the sensors on the clamping members can sense the blood flow through the uterine arteries 16a and 16b which are located a short distance from the vaginal fornix. The above applications are incorporated herein in their entirety by reference.

The procedure may also be monitored by detecting changes in pH of the endometrium lining within the patient's uterus over the treatment period. The pH of the endometrial tissue lining is normally about 6.2 to about 6.8. Upon occlusion of the uterine arteries, the pH of the uterine tissue decreases at least 0.25, usually about 0.5 to about 1.5 pH units from the initial value. As secondary sources begin to provide oxygenated blood to the ischemic tissue, the pH rises back to or near its original levels. The pH may be monitored by placing a pH catheter such as the Zinetics 24 pH catheter from Medtronic, Inc. against the endometrial surface of the uterine wall or into the myometrial tissue of the uterine wall to follow the changes in pH. Either the Zinetics 24 with an external reference electrode secured to the patient's abdomen or thigh, or the Zinetics 24M pH catheter with an internal reference electrode may be employed to monitor the pH. Other methods of monitoring blood flow through the uterine arteries include use of Doppler ultrasound devices and conventional angiographic methods

The present invention may also be utilized to treat conditions which involve or include uterine bleeding, and more specifically to inhibiting or stopping uterine bleeding altogether. As discussed briefly above, there are numerous known conditions which involve or include uterine bleeding. DUB, PPH, and obstetrical, procedures such as caesarian delivery are but a few examples of uterine bleeding which can be inhibited or stopped by methods of the present invention. As described above, the occlusion of the uterine artery and the associated hemostasis in the artery will reduce or completely cut off the blood supply to a portion of the uterus. Simultaneous occlusion of both uterine arteries in a female patient reduces or completely cuts off the blood supply to the uterus, and therefore stops uterine bleeding. Even though the termination of blood flow is short term, there is usually enough thrombus formation to terminate the uterine bleeding at the end of the therapeutic period of time when blood flow is reestablished.

Thus, the present invention extends to a variety of treatment procedures, which can benefit from a reduction in the blood flow to and in the uterus of a patient, including a complete cessation of blood flow. The uterine artery embolization are performed on one or both uterine arteries of the patient. Moreover, the described uterine artery embolization may be performed on one uterine artery and another occluding method may be employed on the other uterine artery such as that described in the co-pending applications Serial No. 09/908,815 and Serial No. 10/107,810 which have been incorporated herein by reference.

While the invention has been illustrated and described herein in terms of occluding uterine arteries for treating uterine disorders, it will be apparent to those skilled in the art that other arteries or body cavities may be occluded or otherwise filled in the same or similar manner to provide therapeutic or diagnostic results. Those skilled in the art will recognize that various modifications and improvements can be made to the invention and that individual features of one embodiment of the invention can be combined with any or all the features of another embodiment of the invention. Accordingly, it is not intended that the invention be limited to the specific embodiments illustrated. It is therefore intended that this invention to be defined by the scope of the appended claims as broadly as the prior art will permit.

Terms such a “element”, “member”, “device”, “sections”, “portion”, “section”, “steps” and words of similar import when used herein shall not be construed as invoking the provisions of 35 U.S.C. §112(6) unless the following claims expressly use the terms “means” or “step” followed by a particular function without specific structure or action.

What is claimed is:

1. A therapeutic procedure for treating a female patient's uterine disorder, comprising non-permanently occluding at least one of the patient's uterine arteries with a bioabsorbable, short lived embolic mass for a therapeutically effective time period.

2. The therapeutic procedure of claim 1 wherein blood flow through the uterine artery system is monitored to ensure re-establishing blood flow through the uterine artery system at the termination of said therapeutically effective time period.

3. The therapeutic procedure of claim 2 wherein the blood flow is monitored by ultrasound.

4. The therapeutic procedure of claim 2 wherein the pH of the uterine wall is monitored during the treatment period.

5. The therapeutic procedure of claim 1 wherein the embolic mass is formed of particulate having a minimum transverse cross section of about 100 to about 2000 micrometers.

6. The therapeutic procedure of claim 5, wherein the particulate has a particle size of about 300 to about 1000 micrometers.

7. The therapeutic procedure of claim 1, wherein the bioabsorbable embolic mass is formed of particulate of a polymeric material selected from the group consisting of polylactic acid, polyglycolic acid or copolymers thereof.

8. The therapeutic procedure of claim 1, wherein the bioabsorbable embolic mass is a swellable particulate material formed of a copolymer of polylactic acid and polyethylene glycol.

9. The therapeutic procedure of claim 8 wherein the weight ratio of polylactic acid to polyethylene glycol is about 95/5 to about 50/50.

10. The therapeutic procedure of claim 8, wherein the copolymer comprises 70% (by wt) polylactic acid and 30% (by wt.) polyethylene glycol.

11. The therapeutic procedure of claim 1 wherein the bioabsorbable embolic mass is a viscous fluid.

12. The therapeutic procedure of claim 1, wherein the occlusion of the uterine artery system includes bilateral occlusion of the uterine artery system.

13. The therapeutic procedure of claim 1, wherein the therapeutically effective time period is about 0.5 to about 48 hours.

14. The therapeutic procedure of claim 1, wherein the therapeutically effective time period is about 1 to about 24 hours.

15. The therapeutic procedure of claim 1, wherein the therapeutically effective time period is about 1 to about 8 hours.

16. The therapeutic procedure of claim 1, wherein the uterine disorder is the presence of uterine fibroids.

17. The therapeutic procedure of claim 1, wherein the uterine disorder is selected from the group consisting of DUB, PPH and bleeding from caesarian section surgery.

18. A therapeutic procedure for treating a female patient's uterine disorder, comprising:
a. advancing a delivery catheter through the patient's vasculature until a distal portion of the catheter is disposed within a desired location of the patient's first uterine artery; and
b. injecting a bioabsorbable, short lived embolic material through the delivery catheter into the desired location of the patient's first uterine artery to temporarily occlude at least a portion of the patient's first uterine artery for a therapeutically effective time period.

19. The procedure of claim 18 including withdrawing the catheter from the patient's first uterine artery and monitoring blood flow through the first uterine artery to ensure reestablishing blood flow through the first uterine artery at the termination of the therapeutically effective time period.

20. The procedure of claim 18 including:
a. advancing a delivery catheter through the patient's vasculature until a distal portion of the catheter is disposed within a desired location within the patient's second uterine artery; and
b. injecting a bioabsorbable, short lived embolic mass through the delivery catheter into the desired location of the patient's second uterine artery to temporarily occlude the patient's second uterine artery for a therapeutically effective time period.

21. An embolic material for temporarily occluding a female patient's uterine artery comprising a bolus formed at least in part of bioabsorbable particulate having a minimum transverse dimension of about 100 to about 2000 micrometer and a suitable carrier.

22. The embolic material of claim 21 wherein the bioabsorbable particulate has a minimum transverse dimension of about 300 to about 1000 micrometers.

23. The embolic material of claim 21 wherein the bioabsorbable particulate is formed at least in part of a polymeric material selected from the group consisting of polylactic acid, polyglycolic acid, polycaprolactone and copolymers, blends and mixtures thereof.

24. The embolic material of claim 21, wherein the bioabsorbable embolic material is swellable particulate.

25. The embolic material of claim 24 wherein the swellable particulate is formed at least in part of a copolymer of polylactic acid and polyethylene glycol.

26. The embolic material of claim 25 wherein the weight ratio of polylactic acid to polyethylene glycol is about 95:5 to about 50:50.

27. The embolic material of claim 25, wherein the copolymer comprises 70% (by wt) polylactic acid and 30% (by wt.) polyethylene glycol.

28. A therapeutic procedure for treating a female patient's uterine disorder, comprising temporarily occluding one or more of the patient's uterine arteries with a bolus of a bioabsorbable, short lived embolic material for a therapeutically effective time period.

29. The therapeutic procedure of claim 28 wherein the embolic material is a solution of a water soluble solvent and a water insoluble, bioabsorbable polymeric solute suitable for intracorporeal deployment.

30. The therapeutic procedure of claim 29 wherein the solvent contains dimethyl sulfoxide.

31. The therapeutic procedure of claim 29 wherein the water insoluble, bioabsorbable polymeric solute is a polymeric material selected from the group consisting of polylactic acid, polyglycolic acid, polycaprolactone and copolymers, blends and mixtures thereof.

32. The therapeutic procedure of claim 28 wherein the embolic material is swellable particulate.

33. The therapeutic procedure of claim 32 wherein the swellable particulate is formed at least in part of a copolymer of polylactic acid and polyethylene glycol.

34. The therapeutic procedure of claim 33 wherein the weight ratio of polylactic acid to polyethylene glycol is about 95:5 to about 50:50.

35. The therapeutic procedure of claim 33 wherein the copolymer comprises 70% (by wt) polylactic acid and 30% (by wt.) polyethylene glycol.

36. The therapeutic procedure of claim 28 wherein the embolic material is a viscous fluid.

37. A therapeutic procedure for treating a female patient's uterine disorder, comprising the steps of:
a. advancing a delivery catheter through the patient's vasculature until a distal portion of the catheter is disposed within a desired location of the patient's first uterine artery; and
b. injecting a first bioabsorbable, short lived embolic material through the delivery catheter into the desired location of the patient's first uterine artery to temporarily occlude at least a portion of the patient's first uterine artery for a therapeutically effective time period.

38. The method of claim 37 including withdrawing the catheter from the patient's first uterine artery and monitoring blood flow through the first uterine artery.

39. The method of claim 37 including the steps of
a. advancing a delivery catheter through the patient's vasculature until a distal portion of the catheter is disposed within a desired location within the patient's second uterine artery; and
c. injecting a second bioabsorbable, short lived embolic mass through the delivery catheter into the desired location of the patient's second uterine artery to temporarily occlude the patient's second uterine artery for a therapeutically effective time period.

40. The method of claim 39 including withdrawing the catheter from the patient's second uterine artery and monitoring blood flow through the second uterine artery.

41. An embolic material for temporarily occluding a female patient's uterine artery comprising a solute of bioabsorbable polymeric material which forms an occluding mass within the patient's artery in a suitable water soluble biocompatible solvent.

42. The embolic material of claim 41 wherein the solvent is dimethyl sulfoxide.

43. The embolic material of claim 41 wherein the bioabsorbable solute is at least in part a polymeric material selected from the group consisting of polylactic acid, polyglycolic acid, polycaprolactone and copolymers thereof.

44. A therapeutic or diagnostic procedure comprising temporarily occluding one or more of a patient's blood vessels with a bioabsorbable, short lived embolic material for a therapeutically effective time period.

45. An embolic material for temporarily occluding a patient's artery comprising a bolus formed at least in part of bioabsorbable polymeric material and a suitable carrier.

46. The embolic material of claim 45 wherein the bioabsorbable polymeric material is in the form of particulate having a minimum transverse dimension of about 100 to about 2000 micrometer.

47. The embolic material of claim 45 wherein the bioabsorbable polymeric material is in the form of particulate having a minimum transverse dimension of about 400 to about 1000 micrometers.

48. The embolic material of claim 45 wherein the bioabsorbable polymeric material is selected from the group consisting of polylactic acid, polyglycolic acid, polycaprolactone and copolymers, blends and mixtures thereof.

49. The embolic material of claim 45 wherein the carrier is a solvent for the polymeric material.

50. The embolic material of claim 49 wherein the solvent contains dimethyl sulfoxide.

51. The embolic material of claim 49 wherein the solute is essentially water insoluble.

52. The embolic material of claim 49 wherein the polymeric material is selected from the group consisting of polylactic acid, polyglycolic acid, polycaprolactone and copolymers thereof

53. The embolic material of claim 45, wherein the bioabsorbable polymeric material is a swellable polymer.

54. The embolic material of claim 52 wherein the swellable polymer is at least in part a copolymer of polylactic acid and polyethylene glycol.

55. The embolic material of claim 53 wherein the weight ratio of polylactic acid to polyethylene glycol is about 95:5 to about 50:50.

56. The embolic material of claim 53, wherein the copolymer comprises 70% (by wt) polylactic acid and 30% (by wt.) polyethylene glycol.

56. The embolic material of claim 45 wherein the bioabsorbable polymeric material is a viscous fluid.

57. A biocompatible solution suitable for intracorporeal deployment comprising a solvent which is relatively soluble in a water based fluid and a bioabsorbable polymeric solute which is relatively insoluble in the water based fluid.

58. The biocompatible solution of claim 57 wherein the solvent is dimethyl sulfoxide.

59. The biocompatible solution of claim 57 wherein the water insoluble, bioabsorbable polymeric solute is selected from the group consisting of polylactic acid, polyglycolic acid, polycaprolactone and copolymers, blends and mixtures thereof.

60. The biocompatible solution of claim 57 containing about 1 to about 35% by weight water insoluble, bioabsorbable polymeric solute.

61. The biocompatible solution of claim 57 containing about 2 to about 20% by weight water insoluble, bioabsorbable polymeric solute.

62. A therapeutic or diagnostic intracorporeal procedure comprising temporarily occluding one or more of a patient's blood vessels with a bolus of a bioabsorbable, short lived embolic material which is a solution of a water soluble solvent and a water insoluble, bioabsorbable polymeric solute.

63. The procedure of claim 62 wherein the solvent contains dimethyl sulfoxide.

64. The procedure of claim 62 wherein the water insoluble, bioabsorbable polymeric solute is a polymeric material selected from the group consisting of polylactic acid, polyglycolic acid, polycaprolactone and copolymers, blends and mixtures thereof.

65. The procedure of claim 62 wherein the embolic material is swellable particulate.

66. The procedure of claim 65 wherein the swellable particulate is formed at least in part of a copolymer of polylactic acid and polyethylene glycol.

67. The procedure of claim 65 wherein the weight ratio of polylactic acid to polyethylene glycol is about 95:5 to about 50:50.

68. The procedure of claim 65 wherein the copolymer comprises 70% (by wt) polylactic acid and 30% (by wt.) polyethylene glycol.

69. The procedure of claim 62 wherein the embolic material is a viscous fluid.

70. An intracorporeal procedure comprising at least partially filling a body cavity or at least partially occluding a body lumen with a bolus of a bioabsorbable, short lived embolic material which is a solution of a solvent which is relatively soluble in a water based fluid and a bioabsorbable polymeric solute which is relatively insoluble in the water based fluid.

71. The procedure of claim 70 wherein the solvent contains dimethyl sulfoxide.

72. The procedure of claim 70 wherein the water insoluble, bioabsorbable polymeric solute is a polymeric material selected from the group consisting of polylactic acid, polyglycolic acid, polycaprolactone and copolymers, blends and mixtures thereof.

73. The procedure of claim 70 wherein the embolic material is swellable particulate.

74. The procedure of claim 73 wherein the swellable particulate is formed at least in part of a copolymer of polylactic acid and polyethylene glycol.

75. The procedure of claim 73 wherein the weight ratio of polylactic acid to polyethylene glycol is about 95:5 to about 50:50.

76. The procedure of claim 73 wherein the copolymer comprises 70% (by wt) polylactic acid and 30% (by wt.) polyethylene glycol.

77. The procedure of claim 73 wherein the embolic material is a viscous fluid.
